(19) United States
(12) Patent Application Publication
(10) Pub. No.: US 2023/0201008 A1
(43) Pub. Date: Jun. 29, 2023

(54) PROSTHETIC-FOOT INSERT AND FORCE-TRANSMISSION ELEMENT, AND SYSTEM COMPOSED THEREOF

(71) Applicant: OTTOBOCK SE & CO. KGAA, Duderstadt (DE)

(72) Inventors: Martin PUSCH, Duderstadt (DE); Christian WILL, Göttingen (DE); Sven KALTENBORN, Duderstadt (DE)

(21) Appl. No.: 17/999,638

(22) PCT Filed: May 25, 2021
(86) PCT No.: PCT/EP2021/063831

§ 371 Date: Nov. 22, 2022

(30) Foreign Application Priority Data
May 26, 2020 (DE) 10 2020 114 021.5

(51) Int. Cl. A61F 2/66

(52) U.S. Cl. CPC A61F 2/66; A61F 2002/6642

(57) ABSTRACT

The invention relates to a system composed of a prosthetic-foot insert (1), comprising: a proximal attachment device (2) for fixing the prosthetic-foot insert (1) on a stump or a prosthetic component; and at least one sole element (3), which is connected to the attachment device (2); wherein the system comprises a plurality of different force-transmission elements (4, 5), which can be fastened to the at least one sole element (3) at at least one fastening region (34, 35) and comprise various distal sole contours (41, 51) and/or various force-transmission regions (42, 52).

The invention relates to a system consisting of a prosthetic-foot insert with a proximal attachment device for securing the prosthetic-foot insert on a stump or a prosthetic component and at least one sole element, which is connected to the attachment device. The invention likewise relates to such a prosthetic-foot insert, to the use of a prosthetic-foot insert as part of a corresponding system, and to a force introduction element for use on a prosthetic-foot insert.

Prosthetic-foot inserts are part of a prosthetic fixture, for example for below-knee amputations. Prosthetic-foot inserts form a distal end section of a prosthesis of a lower extremity and are fastened, for example, to a lower-leg tube or directly to a lower-leg socket. The prosthetic-foot insert is fastened via a proximal attachment device, which is part of the prosthetic-foot insert. The attachment device very frequently has a pyramid adapter, via which multiple settings and orientations of the prosthetic-foot insert can be implemented in relation to a proximal component. The attachment device can furthermore have a bearing block, on which further components of the prosthetic-foot insert are arranged, fastened or mounted. Furthermore, the prosthetic-foot insert has a sole element which either enters directly into contact with the floor or a shoe, or else is surrounded by a prosthesis covering or a cover. The prosthesis covering and the cover serve firstly to protect the technical components of the prosthetic-foot insert and secondly to provide as natural an impression of the finished prosthetic foot as possible. In principle, it is also possible to use the prosthetic-foot insert without a prosthesis covering.

The sole element can have a floor-side contour which is generally curved. Downwardly directed, convex flexions are frequently formed in the heel region and in the ball region in order to enable rolling following a heel strike and rolling over at the end of the stance phase. In addition, contouring can be produced via the cover or the prosthesis covering, which produce a corresponding rolling surface on the underside of the prosthetic foot via a selection of material and thickened portions or weakened portions of material.

A problem with these systems is that only limited adaptability of the prosthetic-foot insert or of the finished prosthetic foot to the individual requirements of the patients or else to changed movement patterns or to changed footwear can be undertaken.

It is therefore the object of the present invention to provide a system with which adaptation of a prosthetic-foot insert to the respective patient can be undertaken more easily.

According to the invention, this object is achieved by a system having the features of the main claim and by a prosthetic-foot insert and a force introduction element having the features of the further independent claims. Advantageous embodiments and developments of the invention are disclosed in the dependent claims, the description and the figures.

The system consisting of a prosthetic-foot insert with a proximal attachment device for securing the prosthetic-foot insert on a stump or a prosthetic component and a sole element, which is connected to the attachment device, makes provision that the system has a plurality of different force introduction elements, which are fastenable to the at least one sole element at at least one fastening region and have different distal sole contours and/or different force introduction regions. The force introduction elements arranged on the sole side of the sole element make it possible to adapt the force introduction point during treading and standing and the course of the force introduction point during walking to the respective user, the respective use conditions and to different activities without a change in the basic setting of the prosthetic foot relative to the remaining prosthetic components being necessary. By provision of a plurality of different force introduction elements with different distal sole contours, the initial force introduction point or force introduction region can be simply changed.

Control of the force introduction during treading, standing and/or walking via the contour of the force introduction point on the sole side is appropriate in particular whenever the different force introduction elements are composed of the same or a homogeneous material. A change in the material thickness results in a different sole contour, as a result of which the manner of the force introduction or load introduction and the position of the load introduction change. Alternatively or additionally, use can be made of force introduction elements with different material properties in certain regions. Force introduction elements with the same contours externally can have harder materials in regions, and therefore, in the event of a loading, the main force introduction takes place at those points or regions which are less easily deformable. As a result, it is possible with force introduction elements which are externally identical and may be colored differently to provide movement characteristics for the respective prosthetic-foot insert or prosthetic foot.

A development of the invention makes provision for the sole element to be configured as a leaf spring or leaf spring section. This leaf spring or the leaf spring section is used as a base or is configured as a base spring for the prosthetic-foot insert and advantageously extends over the entire length of the prosthetic-foot insert.

In an alternative design, the prosthetic-foot insert can have a plurality of sole elements, for example one sole element for the heel region and a second sole element for the forefoot region. If the sole element extends over the entire length of the prosthetic-foot insert, this gives rise to more possibilities for positioning the respective force introduction element at the desired location. The fastening regions can be selected to be larger; there is the possibility of being able to secure a force introduction element in different sections on a fastening region in order thereby to achieve the desired change in the force introduction point. If the sole element is designed as a leaf spring section, it serves to provide an abutment so that the force introduction element can be secured on the prosthetic-foot insert at the desired location.

Advantageously, at least two fastening regions which are arranged separately from one another in longitudinal extent are formed on or fastened to the sole element, in particular one fastening region in the forefoot region or ball region and one fastening region in the heel region, such that, by arranging different force introduction elements in the forefoot region and in the heel region, the overall performance of the prosthetic foot or prosthetic-foot insert can be better adapted to the needs of the patients. With three different force introduction elements for the forefoot region and three different force introduction elements for the heel region, it is possible to set up nine different prosthetic-foot configurations without having to change settings or undertake massive changes in the structure of the prosthetic-foot insert. If a plurality of sole elements are present, the at least two fastening regions are arranged spaced apart from one another in the longitudinal extent of the prosthetic-foot insert, in particular in the front region of the prosthetic-foot insert and in the rear end region of the prosthetic-foot insert.

The fastening regions can also be arranged spaced apart medially and laterally from one another, for example in the forefoot region with different force introduction elements on the big toe section and on the lateral section in the case of a longitudinally divided forefoot spring.

The sole element can be arranged exchangebly on the prosthetic-foot insert, thus making it possible to further influence the force introduction in the elastic prosthetic-foot insert. In addition to influencing the force introduction by adaptation of the sole geometry or sole contour via the force introduction elements or the design of force introduction regions within the force introduction elements, it is possible, by means of a different sole element, to reconfigure a prosthetic foot without there having to be a change in the assignment to other prosthetic components. The new sole element can have a different degree of rigidity; alternatively or additionally, the sole-side contour of the sole element can be different from that of the other sole element. In the case of different sole elements, the fastening regions are preferably designed to correspond or to be identical, and therefore the respective force introduction elements can easily be secured on the respective sole elements.

In one embodiment of the invention, the sole element can be coupled to the attachment device via at least one forefoot spring, at least one heel spring and/or at least one heel pad. Prosthetic-foot inserts have particular damping properties and elastic properties which are achieved by a combination of various spring elements and damper elements.

In order to couple the attachment device to the sole element, various elastic components and damping components can be arranged between them. The attachment device can have, for example, a bearing block which is mounted pivotably via a holder on a forefoot spring which, in turn, is supported on the sole element. On the far side, the attachment device can be supported on the sole element via one or more heel pads, which can be designed as spring-damper devices. The coupling can be undertaken via one or more straps, screws, rivets, hook and loop fasteners, other form-fitting elements or else in an integrally bonded manner by welding or adhesive bonding.

In order to receive and fasten further components, for example the forefoot spring or a heel pad, at least one receiving device via which a form-fitting receiving of the further prosthetic-foot component or system component is implemented can be arranged or formed on the sole element. The receiving device can be in the form of a pocket, insertion opening, undercut, eye or other holder for the component. The assignment of the component to the receiving device can optionally be secured via an aid, such as a screw, clip or a strap.

A development of the invention makes provision that the force introduction elements are arranged or fastened exchangeably on the sole element. The force introduction elements can thus be secured exchangeably on the sole element, for example, in a force-fitting manner by means of a magnet or, for example, a clamping device. Alternatively, form-fitting fastening devices are provided via a hook and loop fastener, plug-in elements, clips, screws, pins or springs which permit destruction-free exchanging and fastening of force introduction elements on the sole element or the sole elements.

The sole contours can differ in shape and/or position of a sole-side force introduction region, wherein the force introduction region can be defined by material elevations or material depressions or by modified materials with differing compressibility or hardness.

The prosthetic-foot insert with a proximal attachment device for securing the prosthetic-foot insert on a stump or a prosthetic component and at least one sole element, which is connected to the attachment device, makes provision that at least one fastening region, to which a plurality of different force introduction elements that have different distal sole contours and/or force introduction regions are fastenable, is arranged or formed on the at least one sole element. The predefined fastening region makes it possible to exchangeably secure the different force introduction elements easily at the locations provided for them using standardized fastening elements or fastening devices. For this purpose, the prosthetic-foot inserts are advantageously equipped or designed as described above.

Different force introduction elements with different properties in respect of the force transfer and force transmission from the underlying surface to the sole element, for example by differently shaped and arranged force introduction regions or different contouring, are advantageously used in order to be secured exchangeably on a prosthetic-foot insert so as to produce a system consisting of a prosthetic-foot insert and the force introduction elements.

Exemplary embodiments of the invention will be explained in more detail below with reference to the figures, in which:

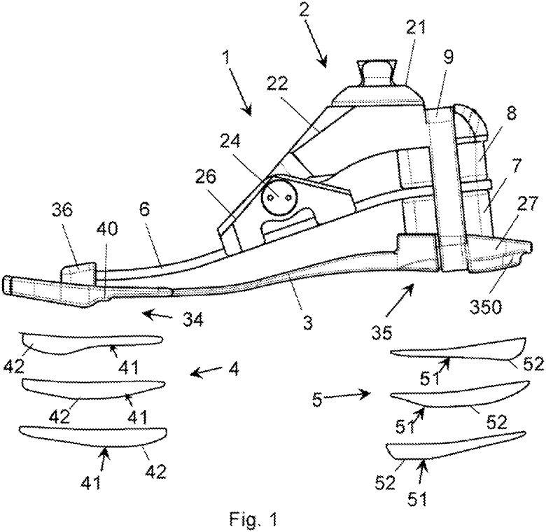

FIG. 1 - shows a system with its components;

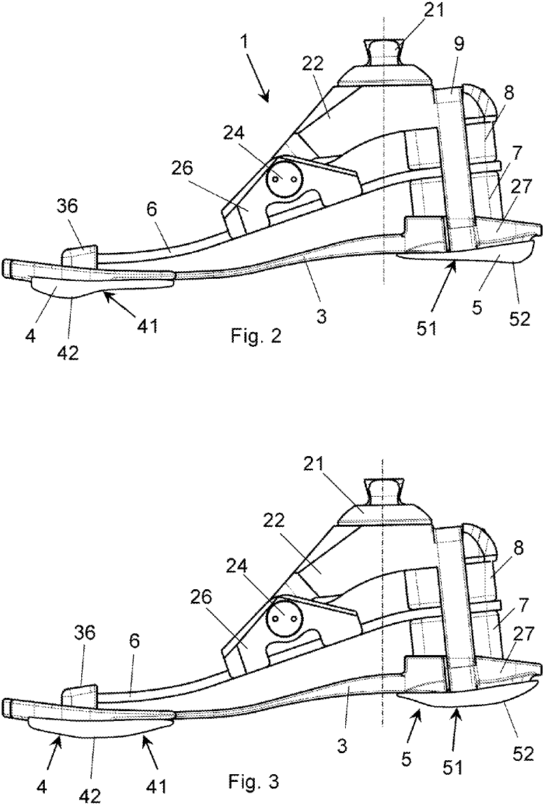

FIG. 2 - shows a first variant with a dynamic configuration;

FIG. 3 - shows a variant with a configuration for a controlled knee flexion;

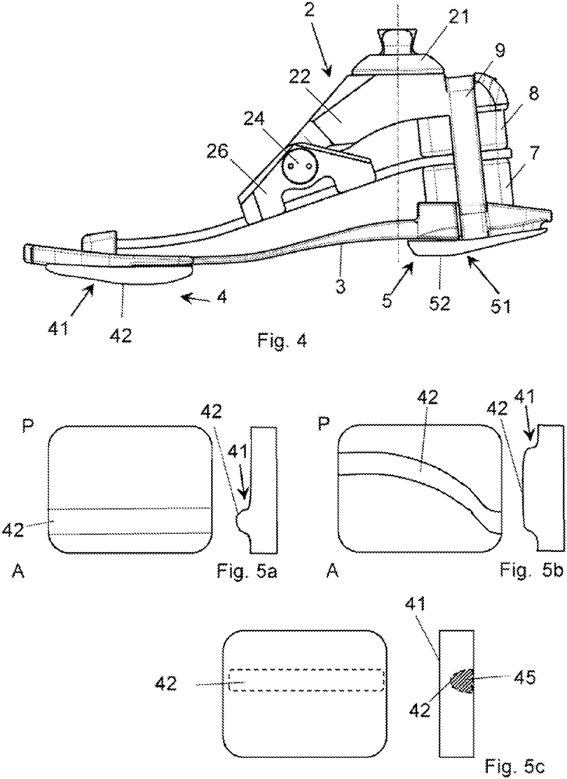

FIG. 4 - shows a variant with a safety-oriented configuration;

FIG. 5 - shows bottom views of force introduction elements;

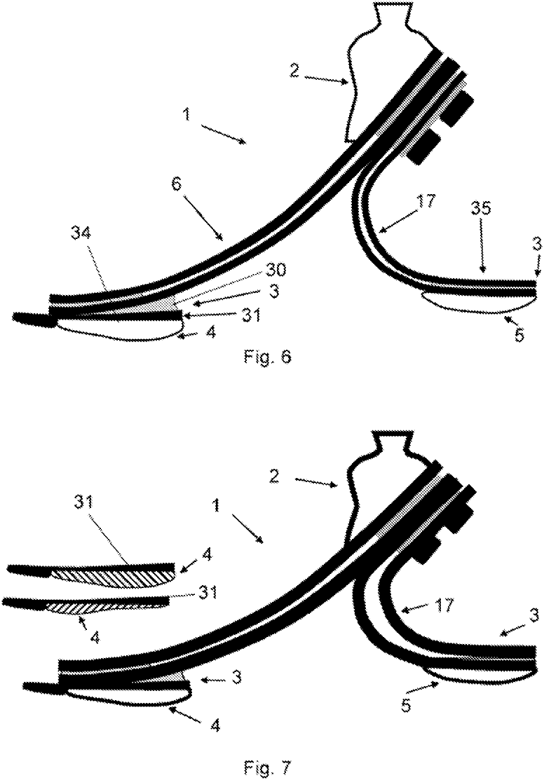

FIG. 6 - shows a variant with two sole elements without a base spring;

FIG. 7 - shows a variant of FIG. 6 with a base force introduction element with exchangeable sections; and

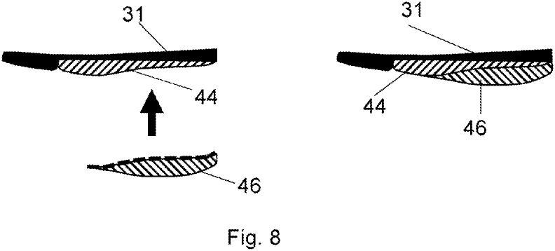

FIG. 8 - shows detailed views of variants of FIG. 7.

FIG. 1 shows, in a side view, a prosthetic-foot insert 1 with an attachment device 2 for fastening to a proximal prosthetic component, not illustrated, for example to a lower-leg tube or a lower-leg socket. The attachment device 2 has a pyramid adapter 21, which is fastened to or formed on a bearing block 22. The bearing block 22 is formed in particular from a dimensionally stable material, for example a light metal. A holder 26 which is connected to a forefoot spring 6 is arranged pivotably about an axis 24 on the front end region of the bearing block 22, as seen in the walking direction. The holder 26 makes it possible for the bearing block 22 to be displaced relative to the forefoot spring 6 during a loading, which may be necessary in the event of deformations during the loading. The rear end of the bearing block 22 is supported on the rear end of a sole element 3 via two heel pads 8, 7. The rear end of the forefoot spring 6 is arranged between the two heel pads 7, 8. The forefoot spring either can be held clamped between the two heel pads 7, 8 or is fastened to at least one of the heel pads 7, 8, for example is fastened or glued thereto in a form-fitting or force-fitting manner.

In order to ensure that the attachment device 2 is assigned to the sole element 3 in the heel region, a strap 9 is guided around the upper side of the bearing block 22 and below the sole element 3 and the length of the strap is set such that the heel pads 7, 8 are prestressed. As a result, the forefoot spring 6 is clamped and held between the two heel pads 7, 8.

In the region of the front end of the forefoot spring 6, receiving devices 36 are arranged on the sole element 3, for example in the form of a pocket with a slot, into which the front end of the forefoot spring 6 is pushed and optionally secured by a fastening element. The forefoot spring 6 is preferably coupled to the sole element 3 so as to be detachable without the sole element 3, the receiving device 36 and/or the forefoot spring 6 being destroyed. As a result, it is possible to change the sole element 3 or to configure the prosthetic-foot insert 1 to meet requirements.

The sole element 3 is designed as a leaf spring and has a first, front fastening region 34 at the front end in the walking direction. The fastening region 34 can be designed as an integral part of the sole element 3. Alternatively, the fastening region 34 can be manufactured separately as an end piece and fastened permanently to the sole element 3. The front fastening region 34 serves for receiving different force introduction elements 4; three thereof are shown below the fastening region 34. At least one form-fitting element 40 is formed or arranged on the fastening region 34 in order to enter into connection with a correspondingly designed form-fitting element, not illustrated, of the respective force introduction element 4. Alternatively thereto, the respective force introduction element 4 can be secured in a force-fitting manner on the sole element 3 at the fastening region 34, for example can be held by clamping or by securing with a magnet. Alternative or additional securings by a clip connection, hook and loop fasteners or by fastening by means of separate fastening elements, such as pins, springs or screws, are likewise possible and provided.

A corresponding fastening region 35 which likewise has form-fitting elements 350 in order to fasten a rear force introduction element 5 is arranged or formed at the rear end of the sole element 3 in the walking direction. In this case too, different types of fastening can be realized in a manner corresponding to the front force introduction elements 4. A receiving device 27 for receiving the lower heel pad 7, into which the heel pad 7 can be inserted, is formed on the upper side of the fastening region 35. The heel pad 7 is thereby secured against lateral displacement and against displacement in and counter to the walking direction. The heel pads 7, 8 are prevented from being able to be detached in the proximal direction via the strap 9 and the prestress.

Below the prosthetic-foot insert 1, three force introduction elements 4, 5 which all have different sole-side contours 41, 51 are in each case shown both in the forefoot region and in the heel region. In an unloaded state, the two upper force introduction elements 4, 5 are provided with force introduction regions 42, 52 lying far apart from each other. The force introduction regions 42, 52 of the middle force introduction elements 4, 5 are planar and each lie further together than in the case of the upper force introduction elements and form a plateau or a relatively large radius of curvature. In the case of the lower force introduction elements 4, 5, the force introduction regions 42, 52 lie closest to each other. The different effects of such a different embodiment of the lower sole contour 41, 51 of the force introduction elements 4, 5 will be explained in more detail below.

FIG. 2 shows a ready fitted first variant of a prosthetic-foot insert 1 with a front force introduction element 4 and a heel-side force introduction element 5, in which the respective force introduction region 42, 52 is substantially linear. During loading of the forefoot region, as seen in the walking direction, the load is introduced relatively far forwards, approximately level with a metatarsophalangeal joint in the case of a natural foot, as a result of which a maximum lever length for introducing a bending moment into the prosthetic-foot insert 1 is obtained. The substantially linear force introduction region 42, 52 makes it possible to absorb or dissipate a very high degree of energy density, and therefore high dynamics can be achieved during walking. Owing to the force introduction region 42 being positioned comparatively far forward and to the linear support with a small supporting surface, increased flexibility during standing is provided.

Corresponding thereto, the heel-side force introduction element 5 is designed with a force introduction region 52 which is arranged as far as possible to the rear, as seen in the walking direction, and which is likewise substantially linear. The rounded, distal force introduction region 52 brings about a concentrated load introduction as far to the rear as possible during treading and rolling following the heel strike. As a result, a maximum lever length is achieved for knee flexion with the orientation of the prosthetic-foot insert 1 relative to a proximal prosthetic component remaining the same. In order to achieve the same effect by adjusting the prosthetic-foot insert 1, the entire prosthetic-foot insert 1 would have to be displaced to the rear or posteriorly relative to a proximal prosthetic component.

In the variant according to FIG. 3, the front or anterior force introduction element 4 is designed with an extensive force introduction region 42 which extends in a length of up to 5 cm in the anterior-posterior direction. As a result, with reference to FIG. 2, the load introduction point will lie posteriorly during rolling, i.e. will produce comparatively little bending moment, low dynamics (absorbs less energy, dissipates less) and more stability since less energy is absorbed and dissipated. In the exemplary embodiment illustrated, the position of the force introduction is limited to the region of the metatarsophalangeal joint in the case of a natural foot and does not extend, as in most other prosthetic-foot inserts, into the front end region of the sole element. This means that, for the initiation of knee flexion in the swing phase, in the case of use of the prosthetic-foot insert 1 with a prosthetic knee joint, the force introduction point is displaced closer to the knee axis. As a result, the initiation of a swing phase is facilitated for the user. This force introduction characteristic permits easier control for the user for initiating the swing phase. With the foot configuration otherwise remaining the same, standing is stabilized, and controlled rolling is made possible, but the bendability or flexibility of the prosthetic-foot insert is reduced and somewhat lower dynamics arise at the forefoot at the end of the stance phase.

The corresponding embodiment of the heel pad 5 in this variant makes provision for a flattened force introduction region 52, in which the load introduction following heel contact and further rolling migrates forward in a controlled manner closer to the knee, i.e. in the direction of a perpendicular line of the pyramid adapter 21. The radius of the sole contour 51 of the rear force introduction element 5 is substantially larger in comparison to the variant according to FIG. 2, and therefore the lever length for a knee-bending moment is reduced while the lower-leg tube rotates forward during rolling of the foot. By means of the slow displacement of the force introduction forward from the force introduction region 52 during rolling, the knee flexion can more easily be controlled than in the case of a narrower force introduction region. The rolling takes place in a more controlled way than in the embodiment according to FIG. 2. In addition, there is lower propulsion from the heel during rolling since, owing to the forwardly migrating force introduction point or the forwardly migrating force introduction line, the moment is reduced because of the smaller lever arm while the axial force remains the same. The forefoot is therefore pushed less strongly into plantar flexion.

In the variant according to FIG. 4, a safer use behavior is produced. The load introduction is limited to a central region which, however, does not extend so far forward as in the variant of FIGS. 2 and 3. The force introduction region 42 ends posteriorly from the metatarsophalangeal joint, as a result of which the lever length for the introduction of the bending moment into the foot during rolling cannot extend as far. As a result, the force introduction point no longer migrates so far forwards, and therefore rolling with small step lengths is facilitated. This increases the safety for less active users. With the foot configuration otherwise being the same, standing becomes more stable and rolling is facilitated because of the small effective foot length. The reduction in the effective foot length results in comparatively low dynamics during rolling and somewhat less flexibility during standing.

A corresponding embodiment of the heel-side force introduction element 5 provides load introduction in a front region of the heel. In the exemplary embodiment illustrated, the force introduction point or force introduction region 52 lies very close to the perpendicular line to the pyramid adapter 21, approximately level with the strap 9. The load introduction is therefore limited to the front region of the heel, as a result of which the lever length for a knee-bending moment after the heel strike is very small. Rolling with small step lengths is facilitated and the safety for less active users is increased because of the good controllability of the rolling behavior. With the configuration otherwise being identical, comparatively safe rolling arises with very little propulsion from the heel.

FIGS. 5a to 5c show schematic bottom views of force introduction elements 4. The embodiment according to FIG. 5a approximately corresponds to the variant which has been explained in more detail with reference to FIG. 2. The force introduction region 42 is limited to a comparatively narrow strip which is illustrated in a cross-sectional view in the right illustration of FIG. 5a. Depending on how far the force introduction region 42 is positioned in the anterior-posterior direction (a-p direction), the stability and dynamics of the foot change. Similarly, the dynamics of the foot are determined by the contour 41 and in particular the radius and the supporting surface of the force introduction region 42.

FIG. 5b illustrates a variant of the force introduction element 4, in which the force introduction region 42 does not run exclusively transversely with respect to the anterior-posterior direction of the force introduction element 4, but rather has a curved shape over the width and in the a-p direction. As a result, the force introduction point or force introduction region 42 after touchdown migrates transversely with respect to the longitudinal extent and along the longitudinal extent of the foot, and therefore the rolling behavior of the prosthetic-foot insert 1 can be further influenced.

FIG. 5c shows a variant of the invention, in which there is a substantially smooth sole contour 41 of the force introduction element 4. The position of the respective force introduction region 42 is realized by different hardnesses of embedded material 45. Where, for example, a narrow, hard web is embedded in the force introduction element 4, which web is surrounded by a flexible, soft material, an identical effect to in the case of a thickened portion downward is produced, as is shown in FIG. 5a.

FIG. 6 shows a variant of the prosthetic-foot insert 1 with the proximal attachment device 2. Two leaf springs, which are combined to form a forefoot spring 6, extend from the attachment device 2 into the front region of the prosthetic-foot insert 1. The forefoot spring 6 has a bend downward, and therefore the front end region of the forefoot spring 6 runs approximately horizontally or is oriented tangentially to the floor. A plate 31 or a spring as a fastening region 34 for a front force introduction element 4 is arranged below the front end of the forefoot spring 6. In the exemplary embodiment illustrated, the plate 31 or spring is fastened permanently to the spring 6 via a connecting means 30, for example an elastomer, such as silicone, or a curable adhesive. Alternatively, the plate 31 is fastened exchangeably to the sole section or sole element 3 of the forefoot spring. The front force introduction element 4 is secured exchangeably on the underside of the plate 31. A bevel or a wedge can be formed or fastened on or to the front end of the plate 31 in order to provide a desired rolling contour, protection for the exchangeable force introduction element 4 or a stop for aligning the force introduction element 4. The wedge or the shoulder is formed in particular from a stiff material, in particular a fiber composite material.

A C-shaped heel spring 17 likewise extends downward from the attachment device 2, with the opening being directed rearward. Analogously to the forefoot spring 6, the heel spring 17 is formed from two leaf spring components running substantially parallel. In the rear end region of the heel spring 17, the spring section runs substantially parallel to the floor, and therefore the sole section 3 is formed by the end piece of the heel spring 17. The rear force introduction element 5 is fastened exchangeably to the heel spring 17 at the fastening region 35 arranged or formed there. A spacing can be ensured between the individual leaf spring components by correspondingly arranged spacer elements or distance elements. The spacing can be formed both in the forefoot spring 6 and in the heel spring 17.

FIG. 7 shows a variant of FIG. 6 which has a substantially identical design to that of FIG. 6. The two force introduction elements 4, 5 which are spaced apart from each other in longitudinal extent, i.e. in the walking direction, are arranged exchangeably on a forefoot spring 6 and a heel spring 17. In the exemplary embodiment which is illustrated, the force introduction elements 4 for securing to the front end of the forefoot spring 6 are provided exchangebly together with the plate 31 as a base. The differently contoured force introduction elements 4 on the underside of the respective plate 31 are either fixedly connected to the plate or arranged exchangebly on the plate. A corresponding embodiment with an exchangeable plate 31 and force introduction elements 5, which are secured or fastened exchangeably thereon, for the heel is correspondingly designed. The plate 31 can have form-fitting elements via which easy exchange is possible by plugging onto and locking to the sole element or the sole elements.

FIG. 8 shows a variant in which a base pad 44 as force introduction element is arranged on the underside of a plate 31. The contour of the base pad 44 can be designed in such a manner that the latter can be used without further modification as a force introduction element with a desired contour. An additional pad 46 which has an attachment contour which corresponds to the outer contour of the base pad is shown below the base pad 44. The additional pad 46 is fastened to the underside of the base pad 44, for example via a form fit, force fit and/or via an integrally bonded fit. Fastening can be undertaken via hook and fleece elements, clip elements, adhesive bonding or magnets or a combination thereof. In the upper right illustration, the force introduction element 4 is illustrated as a combination of base pad 44 and additional pad 46. The rolling contour is produced from a combination of the two pads, wherein the base pad 44 and the additional pad 46 can be formed from different materials. The pads do not have to be produced from a flexible material. It is likewise possible for the respective pad to be differently flexible at different points in order to develop desired rolling properties and optionally shock-absorbing or force-storing properties.

1. A system, comprising:
a prosthetic-foot insert;
a proximal attachment device for securing the prosthetic-foot insert on a stump or a prosthetic component;
at least one sole element connected to the proximal attachment device; and
a plurality of different force introduction elements which are fastenable to the at least one sole element at at least one fastening region, wherein the plurality of different force introduction elements have different distal sole contours and/or different force introduction regions.

2. The system as claimed in claim 1, wherein the at least one sole element is configured as a leaf spring or leaf spring section.

3. The system as claimed in claim 1, wherein the at least one sole element extends over an entire length of the prosthetic-foot insert.

4. The system as claimed in claim 1 wherein the at least one fastening region comprises at least two fastening regions which are arranged separated longitudinally from one another, wherein the at least two fastening regions are each formed on or fastened to the at least one sole element, or wherein the at least two fastening regions are arranged spaced apart from one another longitudinally on the prosthetic-foot insert.

5. The system as claimed in claim 1, wherein the at least one sole element is arranged exchangeably on the prosthetic-foot insert.

6. The system as claimed in claim 1, wherein the at least one sole element is coupled to the attachment device via at least one forefoot spring, at least one heel spring and/or at least one heel pad.

7. The system as claimed in claim 1, further comprising at least one receiving device for form-fitting receiving of a system component, wherein the at least one receiving device is arranged or formed on the at least one sole element.

8. The system as claimed in claim 1, wherein at least some of the plurality of different force introduction elements are fastened exchangeably to the at least one sole element (3).

9. The system as claimed in claim 1, wherein the distal sole contours differ in shape and/or position of a sole-side force introduction region.

10. A prosthetic-foot insert, comprising:
a proximal attachment device for securing the prosthetic-foot insert on a stump or a prosthetic component; and
at least one sole element connected to the proximal attachment device; and
at least one fastening region arranged or formed on the at least one sole element, wherein the at least one fastening region is fastenable with a plurality of different force introduction elements that have different distal sole contours and/or force introduction regions.

11. The prosthetic-foot insert as claimed in claim 10 wherein the at least one sole element is configured as a leaf spring or leaf spring section.

12. A method of using a prosthetic-foot insert as claimed in claim 10, comprising fastening a plurality of different force introduction elements to at least one fastening region of the prosthetic-foot insert.

13. A force introduction element configured for use on a prosthetic-foot insert as claimed in claim 10.

14. The prosthetic-foot insert as claimed in claim 10 wherein the at least one sole element extends over an entire length of the prosthetic-foot insert.

15. The prosthetic-foot insert as claimed in claim 10 wherein the at least one fastening region comprises least two fastening regions which are arranged separated longitudinally from one another, wherein the at least two fastening regions are each formed on or fastened to the at least one sole element, or wherein the at least two fastening regions are arranged spaced apart from one another longitudinally on the prosthetic-foot insert.

16. The prosthetic-foot insert as claimed in claim 10 wherein the at least one sole element is arranged exchangeably on the prosthetic-foot insert.

17. The prosthetic-foot insert as claimed in claim 10 wherein the at least one sole element is coupled to the attachment device via at least one forefoot spring, at least one heel spring and/or at least one heel pad.

18. The prosthetic-foot insert as claimed in claim 10 wherein the distal sole contours differ in shape and/or position of a sole-side force introduction region.
